(19) United States
(12) Patent Application Publication
(10) Pub. No.: US 2019/0053747 A1
(43) Pub. Date: Feb. 21, 2019

(54) METHODS AND DEVICES FOR COLLECTING BLOOD

(71) Applicants: Stanley N. Lapidus, Golden, CO (US); Anthony P. Shuber, Northbridge, MA (US)

(72) Inventors: Stanley N. Lapidus, Golden, CO (US); Anthony P. Shuber, Northbridge, MA (US)

(21) Appl. No.: 16/107,574

(22) Filed: Aug. 21, 2018

Related U.S. Application Data
(60) Provisional application No. 62/548,121, filed on Aug. 21, 2017.

(51) Int. Cl. A61B 5/15; C12Q 1/6806

(52) U.S. Cl. CPC A61B 5/150343; A61B 5/150366; C12Q 1/6806; A61B 5/15003; A61B 5/150473; A61B 5/150229

(57) ABSTRACT

The invention provides methods and devices for drawing blood, in which the blood collected into a container closely resembles the blood as it existed before collection (i.e., as it existed in vivo). In some embodiments this may be achieved by modulating a flow rate of blood into the collection container in order to preserve the integrity of the blood. By modulating the flow rate of the blood, shear forces acting on cells and cellular components in the blood are reduced. This preserves the integrity of such cells and cellular components and facilitates more accurate detection and analysis of low-abundance analytes.

### CROSS-REFERENCE TO RELATED APPLICATIONS

This application claims the benefit of, and priority to, U.S. Provisional Application 62/548,121, filed Aug. 21, 2017, the contents of each of which are incorporated by reference.

### TECHNICAL FIELD

The disclosure relates to methods and devices for collecting blood.

### BACKGROUND

There are many common laboratory analyses performed on blood samples, usually collected from a vein with a needle. Most blood-based analysis targets analytes that are present in abundance in the blood (e.g., cholesterol, white blood cells, hematocrit, etc.). Due to the abundance of the analytes being detected for standard analysis, little or no thought has been given to the integrity of the blood draw as compared to blood in vivo. However, it has recently become possible to assay for blood-borne analytes that are present in low abundance. One example of a low-abundance analyte that has become diagnostically important is circulating, cell-free DNA shed by cancerous or pre-cancerous tissue. With the advent of next generation sequencing technologies it has become possible to conduct a “liquid biopsy” on blood in order to identify and characterize cancer-related nucleic acid. Other uses, including the detection of fetal DNA, are possible as well.

By their nature, low-abundance analytes in a complex sample, such as blood, are subject to signal-to-noise problems and, as a consequence, accurate detection and characterization of target analytes, especially free nucleic acid, has been difficult. A reason for that difficulty may be that the blood sample obtained from a patient through traditional phlebotomy methods does not reflect the integrity of blood in situ. The invention addresses that problem.

### SUMMARY

The invention provides methods and devices for collecting blood in a manner that preserves the in vivo integrity of the blood sample. Methods and devices of the invention allow blood collection in a manner that preserves the molecular integrity of blood components. The result is that the constituents of collected blood mirror those of blood in circulation. Thus, the invention provides more accurate detection and characterization of liquid biopsy samples in which the target for detection typically is nucleic acid in low abundance in the blood.

One particular consequence of methods of the invention is the preservation of the integrity of cells in collected blood. Typically, blood collection creates vortices at the phlebotomy needle tip that result in cell lysis, releasing nucleic acids and other analytes from formerly intact white blood cells into the collection tube. The increased free nucleic acids in the sample make it more difficult to detect low-abundance species and also have an effect on mutant-to-wild-type ratios in the sample. Methods of the invention allow cells to pass from circulation to a collection vessel essentially intact, thus preventing the release of cellular contents from intact circulating cells into the sample. Thus, the ratio of mutant to wild-type DNA in the blood sample is preserved and is more favorable for detecting and analyzing target nucleic acid. For example, methods of the invention are useful for detecting circulating tumor DNA in blood. Circulating tumor DNA typically is present in blood at low levels with respect to wild-type DNA. If cells, such as white blood cells, are ruptured, the resulting mutant-to-wild-type ratio makes detection of the tumor DNA more difficult. Methods of the invention preserve cells, such as white blood cells, resulting in a higher mutant-to-wild-type ratio than would be the case in traditional blood draws.

Methods and devices of the invention use a pump or other such device when drawing blood to maintain laminar flow conditions within the blood that is being drawn. For example, when performing a blood draw, connecting a pump to a collection container allows the pressure to be modulated to maintain a laminar flow. The pump may be provided as an electromechanical pump, syringe pump, bellows pump, vacuum container, etc.

In certain aspects, the invention provides methods for obtaining blood. Preferred methods include the steps of drawing blood from a vein or artery in a manner that substantially preserves the integrity of the blood sample. The cellular components preserved in the blood sample may include one or more of: cell membranes, fragments of cell membranes, proteins, nucleic acids, lipids, enzymes, amino acids, and peptides.

In some embodiments, the cells and cellular components are preserved by modulating the flow rate of blood into a collection container. The collection container may be, for example, a blood bag or a blood collection tube covered by a septum. The flow rate of blood may be modulated into the collection container by an auxiliary device, such as a second vacuum container, a syringe or bellows pump with a constant velocity piston, in fluidic communication with the collection container. The flow rate of blood does not always have to be slowed. For example, the flow rate of blood may be determined by a degree of overall vacuum force acting upon the blood or determined by a pump/piston rate of the auxiliary device.

In related aspects, the invention provides devices for drawing blood that modulate a flow rate of the blood into a collection reservoir to thereby reduce physical forces that would otherwise compromise the integrity of components of the blood, such as white blood cells. The device may include an evacuated container for collecting blood and an auxiliary device to control the influx of the blood into the container. In some embodiments, when the auxiliary device is used, the blood may flow at a lower and constant velocity from the vein into the collection container. The rate of blood flow may be modulated in order to reduce or prevent turbulence in the blood flow at the needle tip. By maintaining a smooth blood flow, hydrodynamic shear forces are significantly reduced and the components of the blood are preserved so that the blood in the collection tube resembles the blood circulating in the body.

One technique for drawing blood uses a blood collection tube such as those sold under the trademark VACUTAINER by BD (Franklin Lakes, N.J.). The blood collection tube is an evacuated tube with a septum covering the open end of the tube, which maintains a vacuum inside of the tube. Blood is initially pulled into the blood collection tube at a high velocity upon puncturing the septum and the flow rate decreases exponentially during the draw as the influx of blood exhausts the vacuum within the tube. Because of abrupt changes in velocity of blood moving from the vein into the needle, turbulence in the blood flow is created. Disparities in the velocity profile of blood as the blood travels through the needle tip cause hydrodynamic shear forces that break apart blood components, such cell walls releasing component from what would otherwise have been stable white cells into the sample. Hydrodynamic shear forces compromise the integrity of the components of blood and negatively impact the usability of the blood sample for downstream blood-based assays. For example, shear forces cause white blood cells to rupture, releasing wild-type DNA that is subsequently collected in the sample. The presence of the wild-type DNA overwhelms the concentration of tumor DNA and prevents the detection of the tumor. The consequence of this effect may result in inaccurate diagnosis.

In certain aspects the invention provides devices and methods for obtaining blood, in which a blood collection container is connected to an auxiliary device such as a pump that modulates flow rate during a blood draw or equilibriates pressure with a collection line. The flow rate may be modulated so as to reduce or prevent abrupt changes in blood flow as the blood moves from the vein and into the needle tip. By reducing or preventing these abrupt changes, the flow velocity profile of blood through the needle may be smooth and hydrodynamic shear forces are significantly reduced. The reduction in shear forces means that the structural properties of the collected blood may be preserved, allowing the blood to exist in the collection container substantially as it did in the vein prior to the blood draw. Because of this, biomarkers in the blood may more easily be detected.

The first evacuated container may be connected to the auxiliary device, such as a pump or other pressure source, in any suitable manner such that the container and the auxiliary device are in fluidic communication. For example, the first evacuated container and the auxiliary device may be connected with tubing. The first evacuated container may be connected to the auxiliary device at any position. For example, the auxiliary device may be connected by tubing to the first evacuated container toward the top or toward the bottom of the container, including at the septum covering the container. The auxiliary device preferably includes a pump that modulates pressure or applies suction. For example, the auxiliary device may include an electromechanical pump, syringe pump, bellows pump, or vacuum source such as an evacuated container, or a source of pressure such as a fluid under pressure (e.g., a pressurized gas).

In certain embodiments, the invention includes a first evacuated collection container connected to a second vacuum container. Preferably, the second container has a greater volume than the collection container such that the shared vacuum of the system will be nearly constant as blood is pulled into the device. The collection container and the second container may be connected via a short piece of tubing. The use of a larger second vacuum container may provide a substantially constant vacuum even as the blood fills the collection tube. The result is that the flow rate of blood into the collection container may be modulated such that it is lower and constant. The lower, constant flow rate of blood from the vein into the collection container may minimize hydrodynamic shear forces at the needle tip as the blood is pulled into the device.

The first evacuated container may be covered by a septum and may be punctured at the septum by a double-ended needle during a blood draw. Each needle of the double-ended needle may be connected by tubing. A blood sample may be taken from a patient by inserting one end of a double-ended needle into the patient (e.g., in the arm) and inserting the other end into the septum of the container to allow blood flow.

In one embodiment, a collection container is connected to a syringe pump or a variation thereof, such as a bellows pump. The syringe pump may be in fluidic communication with the collection container and may regulate the flow rate of blood into the collection container by controlling the movement of the syringe's plunger. The plunger of the syringe may be controlled by a constant velocity motor so that the flow rate of blood into the collection container remains substantially constant despite the influx of blood. Moreover, the operator may increase or decrease the flow rate of blood by regulating the movement of the plunger through the barrel of the syringe.

In another embodiment, multiple collection containers may be connected to a bellows pump or a variation thereof, such as a syringe pump. The bellows pump may be in fluidic communication with the multiple collection containers via a tube and fill adapter, and may regulate the flow rate of blood into the collection containers by controlling the movement of the bellow's piston. The multiple collection containers may be filled in series or in parallel and the containers may vary amongst each other in size, dimension, volume, diameter, or material. The bellows pump may be controlled by a constant velocity motor so that the flow rate of blood into the collection containers remains substantially constant despite the influx of blood. Moreover, the operator may increase or decrease the flow rate of blood by regulating the movement of the piston via its driving mechanism and the bellows pump. By filling multiple containers, a single blood draw may be employed to provide multiple separate containers of blood samples. Each container may be used for detection or other analysis of a separate category of analyte. For example, a first container may be used for analysis involving DNA, a second container may be used for analysis involving RNA, and a third container may be used for analysis involving certain enzymes or metabolites.

### BRIEF DESCRIPTION OF THE DRAWINGS

FIG. 1 shows an evacuated container attached to a second vacuum container.

FIG. 2 illustrates the velocity of blood flow using a standard blood collection tube (solid line) versus the device (dotted line).

FIG. 3 shows a cross sectional view of a needle tip with a turbulent blood flow and a needle tip with a flow rate of blood that is modulated to produce a laminar flow.

FIG. 4 shows an evacuated container attached to a syringe pump.

FIG. 5 shows an evacuated container attached to a bellows pump.

FIG. 6 shows multiple evacuated containers attached to a bellows pump.

### DETAILED DESCRIPTION

The invention provides methods and devices for collecting blood so that the collected blood sample is substantially the same as blood in vivo. By using methods and devices disclosed herein, components of the blood are collected and preserved in a collection container as the components existed prior to being collected and therefore, the blood is a better representation of blood in circulation. The components of blood may include, for example, cells, cell membranes, fragments of cell membranes, DNA, RNA, proteins, enzymes, peptides, or lipids. Preserving the integrity of blood components during collection means that any assay performed on the blood sample will be more likely to yield results that are reflective of the contents of blood in vivo. This is particularly important when one is attempting to identify and/or quantitate low-abundance analytes in blood, such as circulating cell-free tumor DNA.

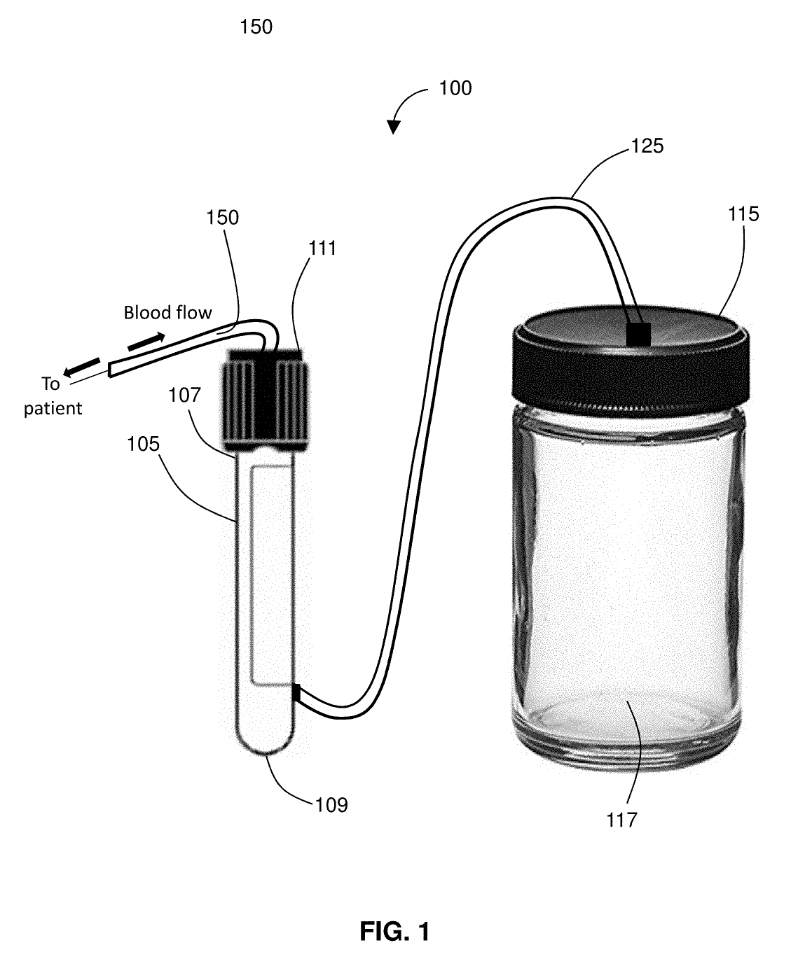

FIG. 1 shows a device 100 for collecting a blood sample. The device 100 includes an evacuated collection container 105 having a closed end 109 and an open end 107 with a septum 111 covering the open end 107 to maintain a vacuum. As shown, collection container 105 is connected to an auxiliary device 115 that is a second container 117. Preferably, the second container 117 may have a larger volume than the collection container 105. For example, the volume of the second container 117 may up to be ten times larger than the collection container 10, up to a hundred times larger, or greater than one hundred times larger. The second container 117 may be held at an initial vacuum that ensures an adequate and essentially constant flow of blood into the collection container 105 during the blood draw. The size and vacuum of the second container 117 may range depending on the size of the collection container 105 and the volume of blood to be drawn. The second container 117 may be cylindrical and may be formed of plastic, such as polyethylene terephthalate (PET). The collection of blood may be terminated either when the desired volume of blood has been collected or when blood hits a hydrophobic membrane in the collection container 105 which allows air to pass but not liquids.

Venipunctures may be performed using the collection container 105 by following a method similar to the methods used for standard blood collection tubes. The blood collection container 105 may be provided with a set of double-sided needles, in which one end of the double-sided needle may be inserted into the vein of a patient and the other side of the needle may then be used to puncture the septum of the blood collection container 105 thereby initiating a flow of blood from the vein and into the needle. The blood may be pulled from the vein into the blood collection container 105 by the vacuum within device 100.

In certain embodiments a blood collection container 105 may be in fluidic communication with an auxiliary device 115 to modulate a flow rate of the blood from the vein into the collection container 105. The auxiliary device 115 may modulate the flow rate of blood by altering the vacuum within the device 100. For example, by reducing the flow rate of the blood into the collection container 105, velocity discontinuities in blood flow may be prevented, and thus, shear forces may be reduced. As a result, the structural integrity of the components of the blood may be better preserved during the blood draw, providing a significant improvement upon the existing blood collection tubes.

As shown, the second container 117 and the collection container 105 may be connected by a tube 125. The tube 125 may be formed from poly ether ether ketone (PEEK) and may attach to on/off valves disposed on the surfaces of the collection container 105 or second container 117. The valves may be disposed wherever it is determined to be most appropriate for maintaining a uniform vacuum without interfering with the collection of blood into the collection container 105. As one trained in the art may appreciate, there are a number of methods for attaching the containers 105 and 117 to one another without departing from the spirit of the invention. For example, the containers 105 and 117 may attach directly via a nozzle located on one end of the second container 117 that then threads into a valve located on a surface of the collection container 105, or vice versa.

Suitable collection containers 105 may preferably be cylindrical and, for example, may be a tube, a flask, or a bottle. The collection container 105 may be formed from a plastic, such as PET, or glass. The collection container 105 may include a transparent or semi-transparent surface such that the volume of blood collected may be visually monitored to terminate the blood draw when a desired volume of blood has been collected. The size of the collection container 105 may range depending on the volume of blood that will be collected, although in general, the tube size may range from 50 to 250 mm with a diameter of 10 to 20 mm. The collection container 105 may be treated with heparin, as is standard in the art, to provide the container 105 with blood clotting properties. Moreover, additives, such as ethylenediaminetetraacetic acid (EDTA), may be included in the container 105 to enhance the blood preservation features of the device.

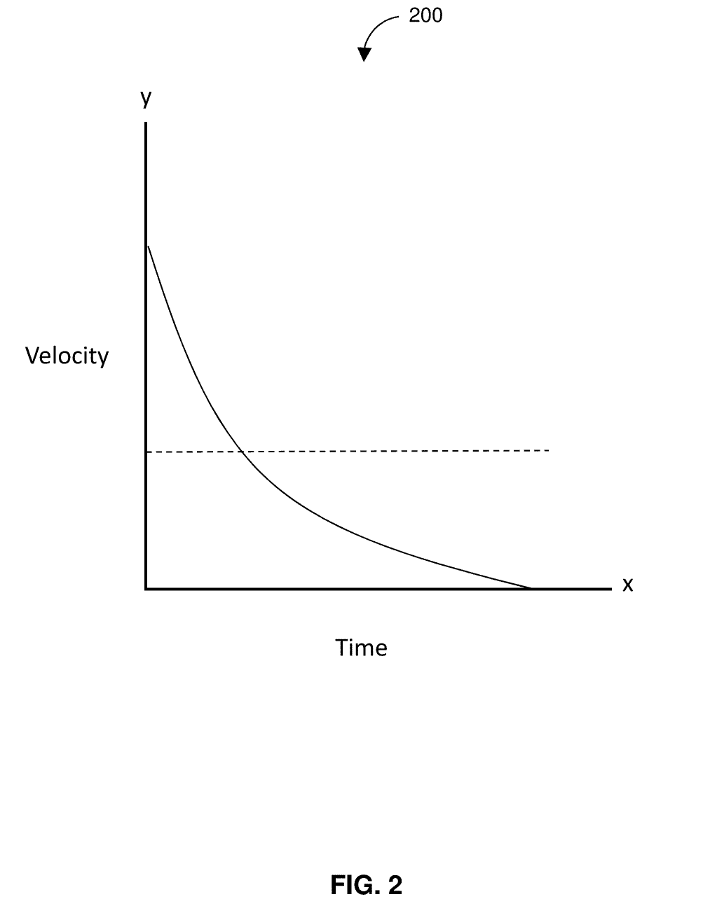

FIG. 2 illustrates a velocity of blood flow using a standard blood collection tube (solid line) versus the device (dotted line).

Existing blood collection tubes create a high velocity blood pull from the vein into the blood collection tube when the septum is initially punctured. The velocity profile of blood moving from the vein and into the blood collection tube is typically that of a single decaying exponential curve (illustrated by a solid line). The blood flow rate into the blood collection tube is at its highest when the septum is pierced and rapidly goes to zero as the tube vacuum is depleted by the influx of blood.

In contrast, device 100 may maintain a lower and substantially constant flow rate of blood (dotted line). Replacing the exponential flow with a substantially constant flow rate is a way to move the same volume in the same time but at approximately 2.5× lower peak flow rate.

Abrupt changes in blood movement result in hydrodynamic shear forces at the needle tip. When the septum of a standard blood collection tube is initially punctured, a high vacuum force pulls the blood from the vein and into the needle at a high velocity. The high velocity movement of blood into the needle tip relative to the blood flow in the vein creates turbulence in the blood flow.

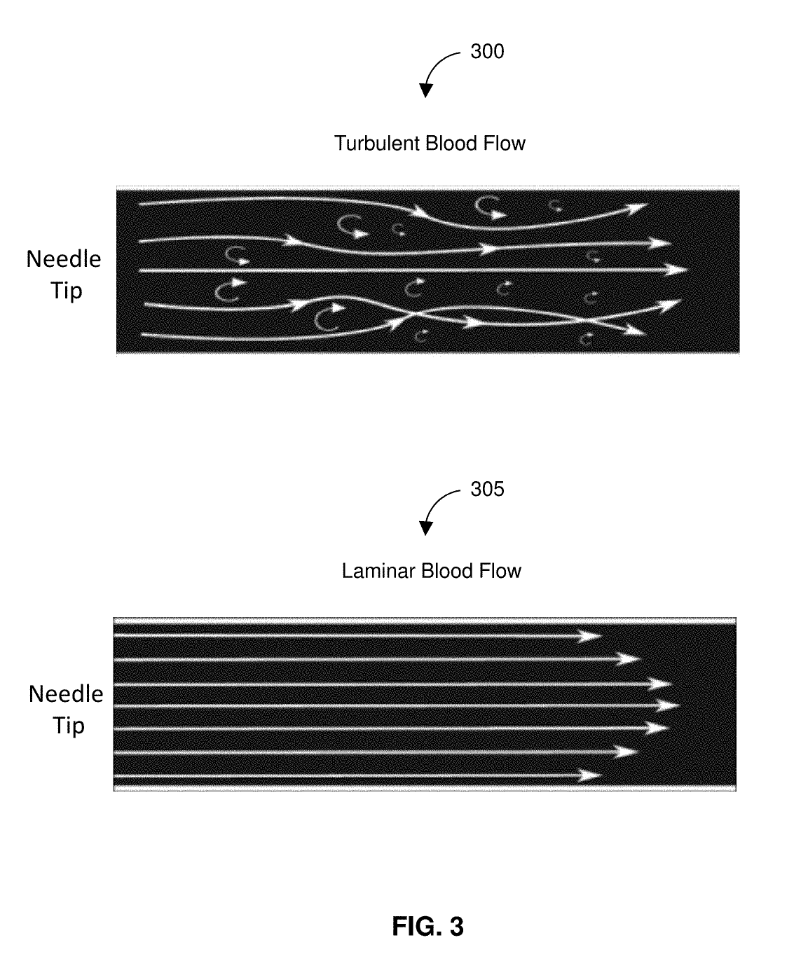

FIG. 3 shows a cross sectional view 300 of a needle tip with a turbulent blood flow and cross sectional view 305 of a needle tip with a flow rate of blood modulated to produce a laminar flow through the needle. Cross sectional view 300 of turbulent blood flow shows how high velocity movement of blood into the needle relative to the vein creates turbulence in blood flow.

The turbulent flow is greatest at the needle tip because of the disparity in flow rate of blood circulating through the vein versus the needle. When the flow is turbulent, blood eddies at the needle tip causing hydrodynamic shear forces that compromise the structural integrity of components of the blood.

In some embodiments the invention may employ an auxiliary device 115 that when used in conjunction with a collection container 105, draws blood at a reduced and substantially constant flow rate. A reduced flow rate of blood from the vein may prevent abrupt changes in blood flow at the needle tip. By reducing the flow rate, and avoiding abrupt changes in blood flow from the vein to the needle, the blood flow profile may be made laminar rather than turbulent. Auxiliary device 115 may be, for example, a second vacuum container, a syringe or bellows pump with a constant velocity piston, in fluidic communication with the collection container. In other embodiments, the flow rate of blood does not always have to be slowed. For example, the flow rate of blood may be modulated by a degree of overall vacuum force acting upon the blood created by the auxiliary device or determined by a pump/piston rate of the auxiliary device.

Cross sectional view 305 of laminar blood flow illustrates how by reducing the flow rate of blood into a collection container 105, hydrodynamic shear forces may be reduced. The operator of the device may further reduce hydrodynamic shear by drawing the volume of blood at constant velocity over a longer time.

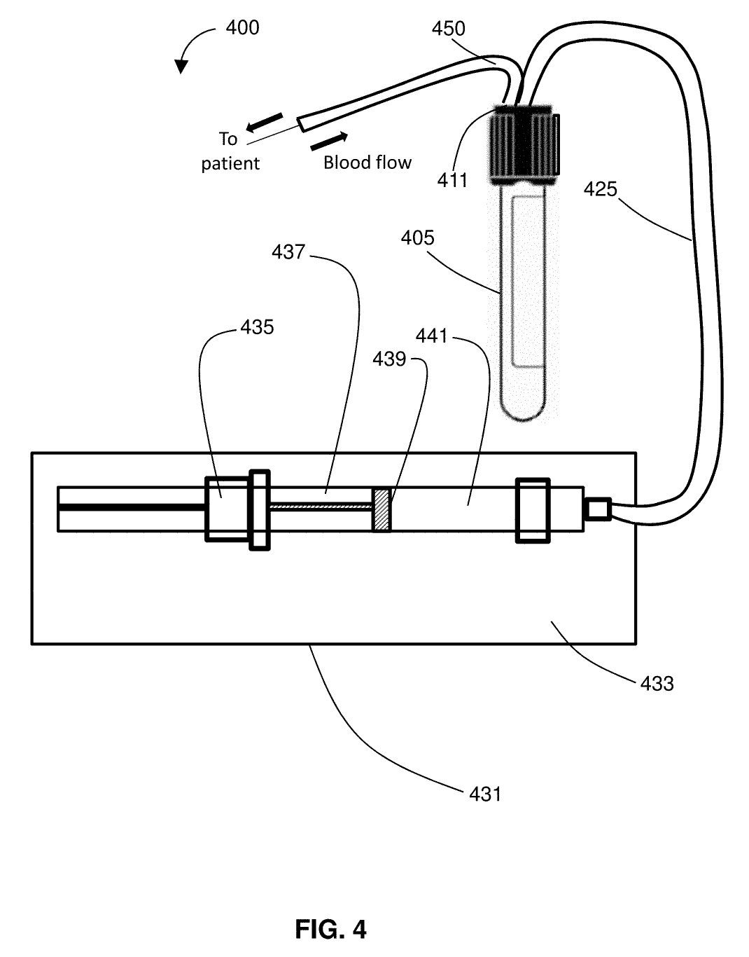

FIG. 4 shows a device 400 for collecting a blood sample in which the influx of blood into the collection container 405 is regulated by an auxiliary device 431. Any suitable auxiliary device that applies suction or a pressure deficit to the device 400 may be used. For example, the auxiliary device 431 may include an electromechanical pump or other such device. In the depicted embodiment, the auxiliary device 431 includes a syringe pump 433. The syringe pump 433 may be in fluidic communication with a collection container 405 such that when the septum 411 of the collection container 405 is punctured during the blood draw, the syringe pump 433 may modulate the flow rate of blood drawn into the collection container 405 by regulating the vacuum throughout the device 400. Although illustrated with a bellows pump, device 400 may employ a bellows pump, a second evacuated container, or any other device suitable to modulate the flow of blood into the collection container 405.

In one example, a double-ended needle 450 may be inserted into a patient and into septum 411 of the collection container 405. Alternatively, the double-ended needle 450 may be inserted into other aspects of device 400 such that it is in fluid communication with collection container 405. The syringe pump 433 may modulate the flow of blood from a vein of the patient and into collection container 405.

The syringe pump 433 may include at least one syringe 441 and a reciprocating drive mechanism 435 able to be attached to the at least one syringe 441. The drive mechanism 435 may regulate the movement of at least one plunger 439 relative to the barrel 437 of the at least one syringe 441. The syringe 441 may be connected to the collection container 405 by a tube 425, in which the collection container 405 functions as a reservoir for the blood withdrawn from the patient's arm while the syringe pump 433 modulates the blood flow into the collection container 405 by controlling the vacuum via the movement of the plunger 439. In this example, the blood flow rate is modulated by the degree of overall vacuum throughout the device 400. During a blood draw, any depletion in vacuum caused by the influx of blood into the collection container 405 may be offset by the outward movement of the plunger 439 within the syringe 441. The volume of blood withdrawn from the patient's arm per unit of time may be controlled by the operator by adjusting the vacuum to any desired level consistent with acceptable draw time and volume. The syringe pump 433 may include a plurality of syringes 441 so that when the plunger 439 reaches the bottom of the barrel 437 of the syringe 441 the blood flow rate will not be interrupted. The syringe pump 433 may be connected to and operated by a computer controlled stepper motor.

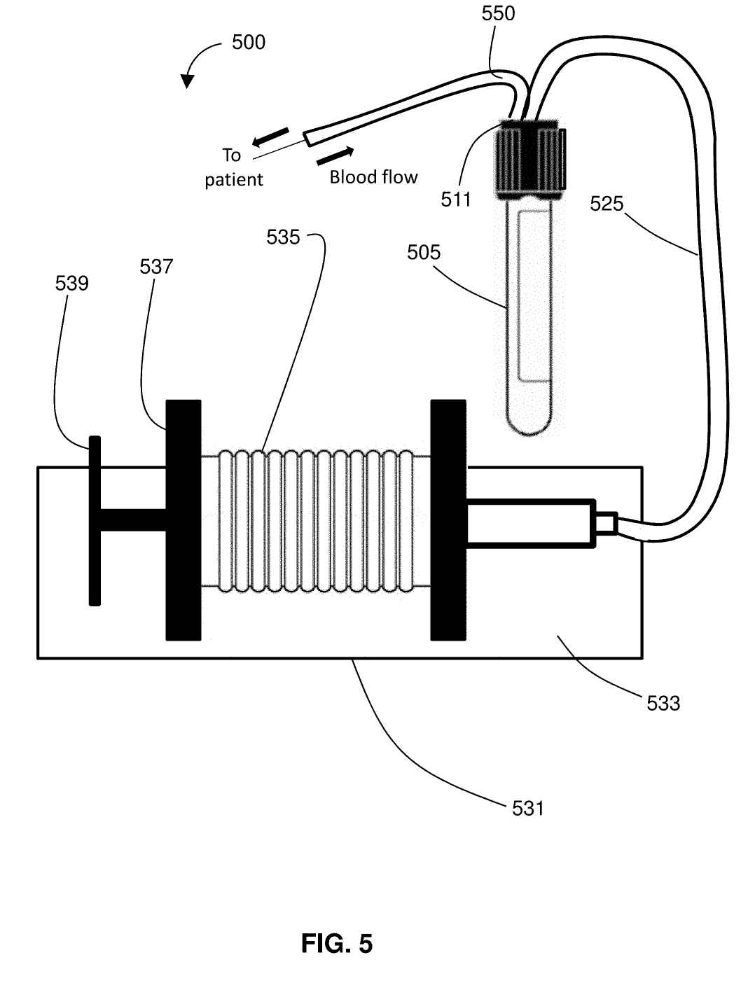

FIG. 5 shows a device 500 for collecting a blood sample in which the influx of blood into the collection container 505 is regulated by an auxiliary device 531, which modulates pressure of the influx of blood into the collection container 505. The auxiliary device 531 may be provided by any suitable pump device. In the depicted embodiment, the auxiliary device 531 includes a bellows pump 533. Device 500 includes a blood collection container 505 connected to a bellows pump 533. The bellows 535 may be attached at one end by a moveable piston 537 that is connected to a drive mechanism 539.

In one example, a double-ended needle 550 may be inserted into a patient and into septum 511 of the collection container 505. Alternatively, the double-ended needle 550 may be inserted into other aspects of device 500 such that it is in fluid communication with collection container 505. The syringe pump 533 may modulate the flow of blood from a vein of the patient and into collection container 505.

As the piston 537 is moved in a direction away from the bellows 535 by the drive mechanism 539, the bellows 535 may expand and create a vacuum within the device 500 resulting in an influx of blood into the collection container 505. The volume and velocity of blood collected from the patient may be controlled by the operator by modulating the rate at which the piston 537 is moved. The bellows pump 533 may be connected and operated by a computer controlled stepper motor. The collection of blood may be terminated either when an observed target volume of blood has been drawn or when blood hits a hydrophobic membrane in the collection container which allows air to pass but not liquids.

The device 500 may include additional features to enhance the device's use for drawing blood. For example, the device 500 may include protective casings around the septum 511 to protect the user when attaching a collection container 505 to a needle inserted in the patient's arm. The device 500 may include sensors and alarms that detect hemolysis within the collection container 505 and alert the operator to make adjustments to the flow rate of blood. The device 500 may include sensors that alert the operator when a predetermined amount of blood has been withdrawn from the patient.

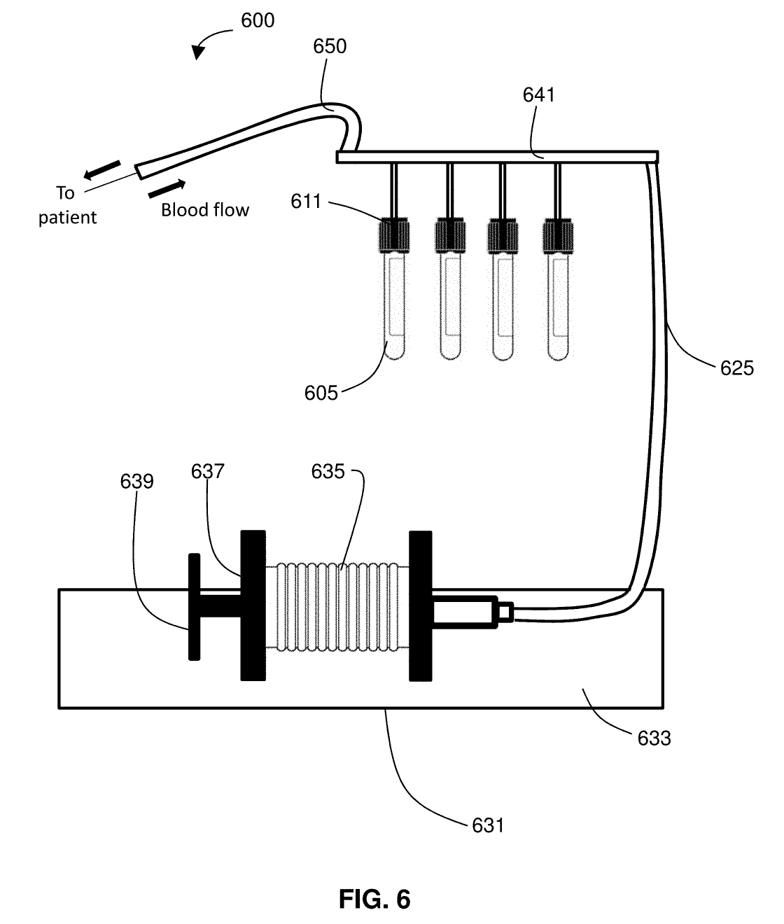

FIG. 6 shows a device 600 for collecting a blood sample that includes multiple evacuated containers 605 attached to a pump 633. Any suitable pump may be used such as an electromechanical pump. In the depicted embodiment, pump 633 is a bellows pump. Although illustrated with a bellows pump, device 600 may employ a syringe pump and syringe, a second evacuated container, or any other device suitable to modulate the flow of blood into the collection containers 605. When collecting blood, device 600 may fill the multiple evacuated collection containers 605 in a series or parallel configuration. Any number of collection containers 605 may be filled in series or in parallel, for example, at least two, at least eight, at least twelve, or more than twelve. In addition, the containers 605 may vary amongst each other in size, dimension, volume, diameter, or material and be filled in series or in parallel depending on the operator's configuration. By filling multiple containers 605, device 600 allows a single blood draw to provide multiple separate containers 605 of blood, in which each container 605 may be used for detection or other analysis of a separate category of analyte. For example, a first container 605 may be used for analysis involving DNA, while a second container 605 may be used for analysis involving RNA, and a third container 605 may be used for analysis involving certain enzymes or metabolites.

In one example, a double-ended needle 650 may be inserted into a patient and into fill adapter 64, connected to each septum 611 of the multiple collection containers 605. Alternatively, the double-ended needle 650 may be inserted into other aspects of device 600 such that it is in fluid communication with multiple collection containers 605 via fill adapter 641.

When collecting a blood sample, pump 633 may regulate the influx of blood into the multiple collection containers 605. As shown, device 600 includes blood collection containers 605, which are connected to a pump 633 via tube 625 and fill adapter 641. The bellows 635 may be attached at one end to a moveable piston 637 that is connected to a drive mechanism 639. As the piston 637 is moved in a direction away from the bellows 635 by the drive mechanism 639, the bellows 635 may expand and create a vacuum within the device 600 resulting in an influx of blood into the collection containers 605, which may be filled in a series or parallel configuration. The volume and velocity of blood collected from the patient may be controlled by the operator by controlling the rate at which the piston 637 is moved. The pump 633 may be connected and operated by a computer controlled stepper motor. The collection of blood may be terminated either when an observed target volume of blood has been drawn across the collection containers 605 or when blood hits a hydrophobic membrane in a specified collection container which allows air to pass but not liquids.

The device 600 may include additional features to enhance the device's use for drawing blood. For example, the device 600 may include protective casings around septums 611 to protect the user when attaching collection containers 605 to a needle inserted in the patient's arm. The device 600 may further include sensors and alarms that detect hemolysis within the collection container 605 and alert the operator to make adjustments to the flow rate of blood. The device 600 may include sensors that alert the operator when a predetermined amount of blood has been withdrawn from the patient.

Methods and devices of the disclosure may be used to improve liquid biopsies. Additionally, the methods and devices of the disclosure may be used to study circulating tumor DNA (ctDNA) in real time before and during treatment. Because the methods and devices provide a non-invasive opportunity to study ctDNA, blood samples may be collected at discrete times during treatment and the information provided by the ctDNA may be used to track mutations, such as epigenetic alterations and other forms of tumor-specific abnormalities, over the course of a patient's treatment. Clinicians may use this information to formulate and adjust tumor-specific therapies for individual patients.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### Equivalents

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A device for collecting a blood sample, the device comprising:
a collection container having a structure that substantially preserves the integrity of cells and cellular components in a blood sample when the blood is drawn into the collection container.

2. The device according to claim 1, wherein the cellular components includes one or more of the following: cell membranes, fragments of cell membranes, proteins, nucleic acids, lipids, enzymes, amino acids, peptides.

3. The device according to claim 1, wherein the structure is an auxiliary device in fluidic communication with the collection container to modulate flow into the collection container during a blood draw.

4. The device according to claim 1, wherein the collection container comprises a blood collection tube covered by a septum.

5. The device according to claim 1, wherein the collection container comprises a blood bag.

6. The device of claim 3, wherein the auxiliary device comprises a second container.

7. The device of claim 3, wherein the auxiliary device comprises a pump.

8. The device according to claim 3, wherein the auxiliary device modulates a flow of blood into the container so as to reduce shear forces.

9. The device of claim 3, wherein the collection container comprises a transparent or semitransparent surface to allow the volume of collected blood to be monitored.

10. A method for obtaining a blood sample, the method comprising the steps of:
withdrawing blood from a vein or artery in a manner that substantially preserves the integrity of cells and cellular components in the blood sample.

11. The method according to claim 10, wherein the cellular components include one or more of: cell membranes, fragments of cell membranes, proteins, nucleic acids, lipids, enzymes, amino acids, and peptides.

12. The method of claim 10, further comprising modulating a flow rate of blood into a collection container to preserve the integrity of the cells and cellular components.

13. The method of claim 12, wherein the collection container comprises a blood collection tube covered by a septum.

14. The method of claim 12, further comprising modulating the flow rate of blood into the collection container using an auxiliary device in fluidic communication with the collection container.

15. The method of claim 14, wherein the flow rate is modulated so as to reduce shear forces acting on the cells and cellular components during collection.

16. The method of claim 14, wherein the auxiliary device comprises a second container.

17. The method of claim 14, wherein the auxiliary device comprises a pump.

18. The method of claim 14, wherein the collection container comprises multiple blood collection containers and wherein withdrawing blood further comprises filling the multiple blood collection containers in series or in parallel.

19. A method for collecting blood, the method comprising the steps of:
withdrawing blood from a vein or an artery in a manner that preserves an in vivo ratio of mutant-to-wild-type nucleic acids.

20. A sample for medical analysis, the sample comprising a container having within it a volume of blood in which a ratio of mutant-to-wild-type nucleic acid is substantially identical to that of blood in vivo.
